(19) United States
(12) Patent Application Publication
(10) Pub. No.: US 2007/0249980 A1
(43) Pub. Date: Oct. 25, 2007

(54) Device for Fixing on the Skin a Catheter Exiting a Cutaneous Emergency Site

(75) Inventors: Jean-Luc Carrez, Ecouen (FR); Valery Dalle, Gouvieux (FR); Jean-Philippe Mullard, Ermont (FR); Mathias Schleuniger, Paris (FR)

Correspondence Address: LEVINE & MANDELBAUM, 444 MADISON AVENUE, 35TH FLOOR, NEW YORK, NY, 10022, US

(73) Assignee: Vygon, Ecouen (FR)

(21) Appl. No.: 11/576,959

(22) PCT Filed: Oct. 10, 2005
(86) PCT No.: PCT/FR05/02486

§ 371 Date: May 23, 2007

(30) Foreign Application Priority Data
Oct. 14, 2004 (FR) 0410855
Oct. 6, 2005 (FR) 0510220

(51) Int. Cl. A61F 13/02

(52) U.S. Cl. 602/47

(57) ABSTRACT

A device for fixing on the skin a catheter exiting a cutaneous emergency site has a flexible pad with an adhesive underside, a side slot starting from the edge of the pad and terminating in a hole which runs through the pad, the slot being sufficiently large for the pad to be laterally slid through the slot onto the catheter exiting the site until the hole is located on the site. The hole is located at the base of a housing in the upper surface of the pad. The pad has a foldable side tab including a cap adapted to be elastically wedged in the housing above the catheter when the tab is folded on the pad.

The invention concerns a device to secure onto the skin a catheter exiting from a cutaneous emergence site.

Many devices have been proposed to secure onto the skin a catheter exiting from such a puncture site.

Such devices are described, for example, in publications EP 078 041, WO 95/20 415 (=EP 0 541 189), DE 411 282 (-EP 0 515 807), U.S. Pat. No. 3,918,446, U.S. Pat. No. 4,324,237, EP 0 931 560, U.S. Pat. No. 5,690,616, AU 589 549, U.S. Pat. No. 4,364,368, and U.S. Pat. No. 4,517,971.

Devices have also been proposed to retain a catheter on the skin of a patient by passing it longitudinally (U.S. Pat. No. 4,397,647) or transversally (U.S. Pat. No. 6,572,588) in a passage formed on a pad fixed to the skin, and holding it there by a mobile plug adjoining the pad and designed to be led up to a shaped portion of this passage above the catheter, but these devices have not been designed to cover an emergence site in order to retain the catheter at the exit from the site.

This present invention aims to supply a device based on a pad and a plug, that is inexpensive to manufacture, of minimum dimensions, easy and reliable to use, comfortable for the patient, and designed to secure a catheter at the exit from a cutaneous emergence site.

This device, which is essentially composed of a flexible pad whose underside has been rendered adhesive, which is equipped with a slot (7) starting from the edge of the pad and ending at a hole which traverses the pad from side to side, this slot being sufficiently wide so that the pad can be slid laterally via the slot onto the catheter exiting from the emergence site until the said hole can be placed on the site itself, is characterised in that the said hole is located at the bottom of a recess present on the top surface of the pad, and in that the pad includes a pliable lateral tongue which includes a plug designed to be trapped elastically in the said recess on top of the catheter when the tongue is folded onto the pad.

The emergence site, as well as the catheter at the exit from this site, are protected by the plug when the latter is trapped in the recess. In preferred implementations, the device also has one or more of the following characteristics:
the recess is circular and open laterally at the side of the tongue, so as to accommodate the part of the tongue that immediately precedes the plug when the tongue is folded;
the plug projects above the recess when it is trapped in the latter;
the plug is split so as to be elastically deformable and so that it will be trapped in the recess;
the recess, the tongue and the plug are created by moulding with the pad;
the pad includes a channel formed on top of the pad in order to accommodate the catheter, by elastic trapping, at the exit from the hole;
the said channel (8′) is on a downward slope as far as the edge of the pad in order to guide the catheter until it makes contact with the skin;
the channel extends in a straight line from the edge of the recess as far as the edge of the pad;
the channel is located in extension to the said slot;
the channel lies perpendicularly to the said slot;
the recess is formed in a thickened area of the pad;
the pad has a base surface facing upwards, on which ribs form the said slot and the said channel between them;
the pad has several channels, and includes retention resources to allow the catheter to be wound onto the periphery of the pad, so that the catheter, at the exit from one of the channels, can be wound by following a part of the edge of the pad until it can be inserted, in the reverse direction, in another of the channels which again brings it above the site and it then passes into the slot or into one of the channels as far as the periphery of the pad;
the retention resources include side-walls which extend the bottom face of the pad laterally by following its contour between the exits from the channels and from the slot in the pad;
the retaining side-walls are raised in relation to the pad;
the side-walls are walls which are sloped upwards so as to retain the catheter;
the retention resources include ribs formed on the bottom face of the pad and whose extremities project outwards in relation to the base face.

The following is a description of preferred but not limiting implementations of devices according to the invention, with reference to the figures of the attached drawings, which are enlarged in relation to reality, and on which:

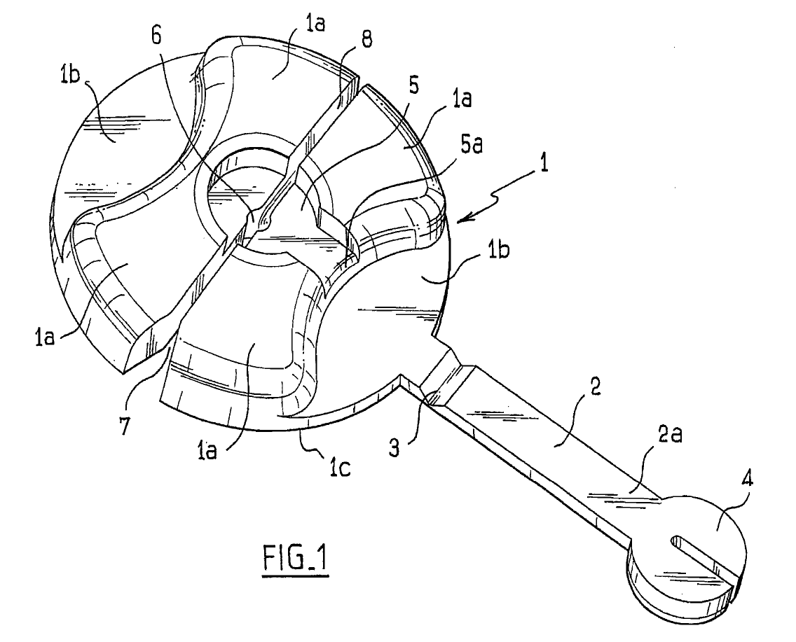

FIG. 1 is a view in perspective of the device in the open state;

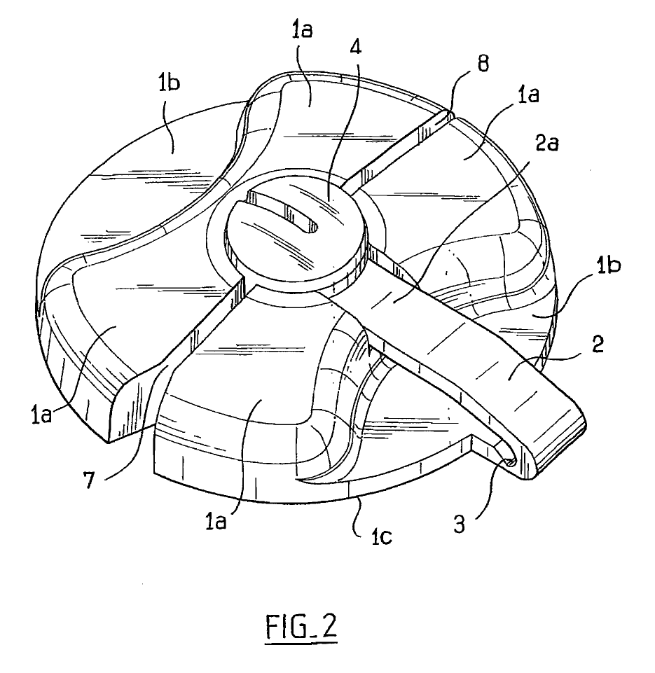

FIG. 2 is a view in perspective of the device in the closed state;

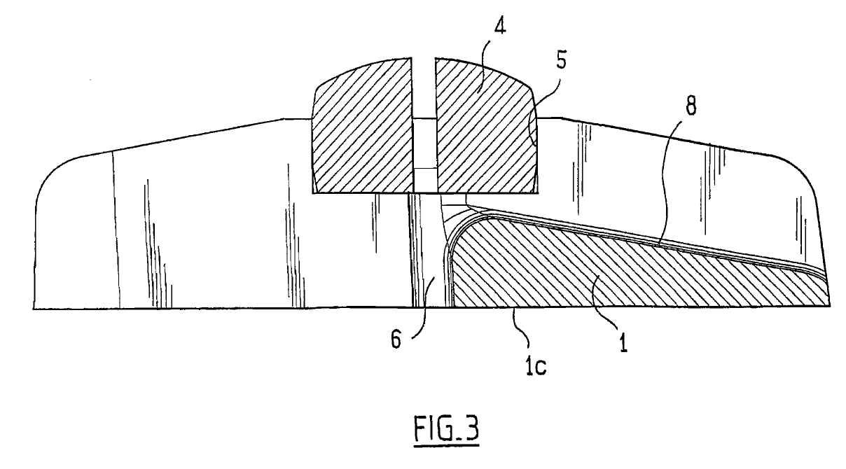

FIG. 3 is a view in section of the device in the closed state;

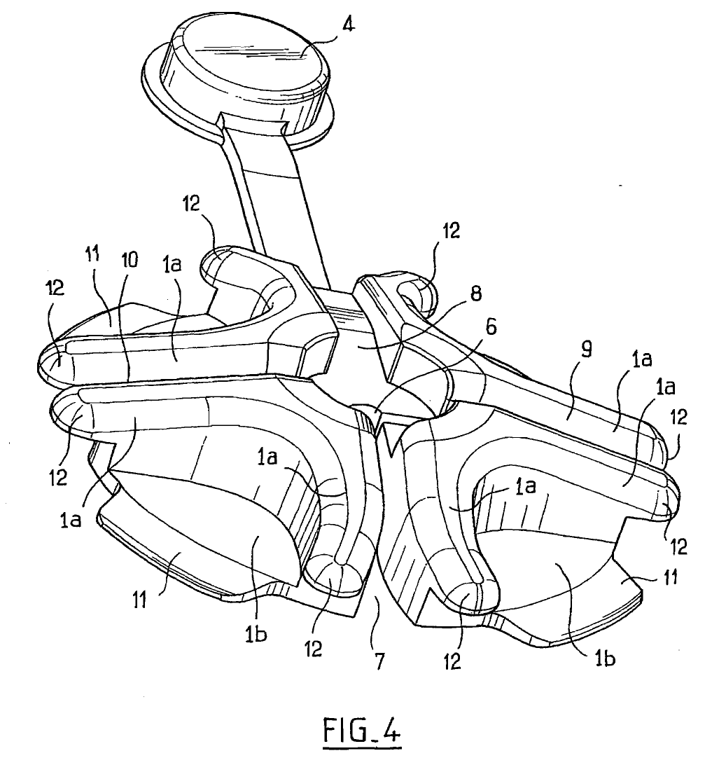

FIG. 4 is a view in perspective of an implementation variant of a pad which has been adapted so that the catheter can be wound onto at least part of the perimeter of the pad;

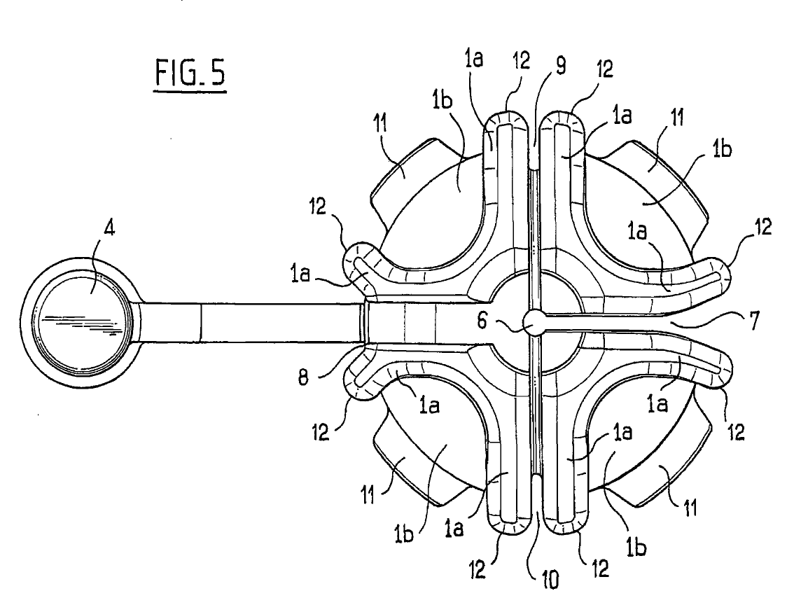

FIG. 5 is a view from above of the implementation variant of FIG. 4;

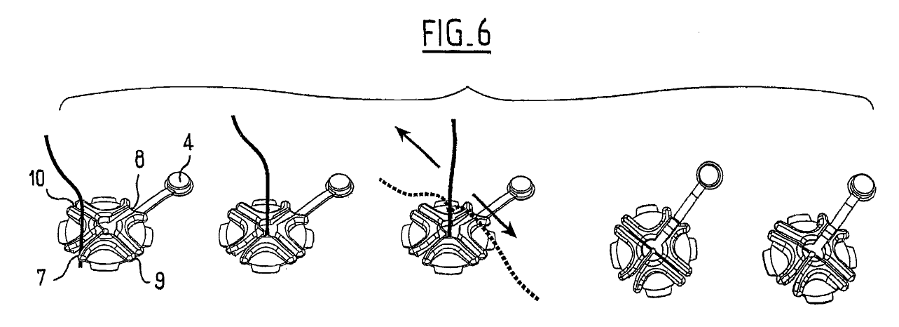

FIG. 6 is a diagram of the procedure for fitting the pad of FIG. 4, and

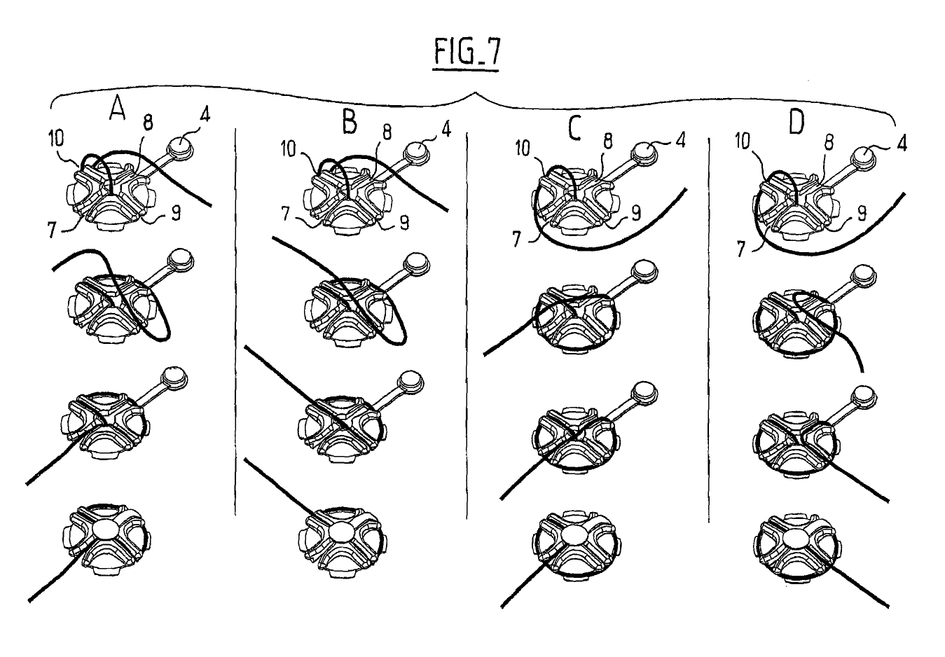

FIG. 7 diagrammatically shows different options for the retention of the catheter on a pad according to FIG. 4.

The device shown in FIGS. 1 to 3 is a circular pad (1) created by moulding with a lateral tongue (2) that is hinged to the pad by a thinned notch (3) to form a fold line allowing the tongue to be folded onto the pad until a plug (4), created by moulding at the extremity of the tongue, arrives above an open recess (5) formed in the body of the pad around a hole (6) which traverses the pad from side to side.

The pad includes ribs in relief (1a) on a base face (1b) facing upwards, with these ribs forming between them a rectilinear slot (7) which connects the edge of the pad to the hole (6) and which is flared at its entrance, and a rectilinear channel (8) which, in the case illustrated, is located on the other side of the hole (6) as an extension to slot (7).

The slot (7) is dimensioned to allow the pad to slide laterally on the catheter via the said slot, until the hole (6) comes level with the puncture site, and the channel (8) is dimensioned to retain the catheter by elastic trapping when it is bent onto the pad at the exit from the hole. Preferably, this channel is on a downward slope as far as the edge of the pad in order to guide the catheter until it makes contact with the skin.

The recess (5) is dimensioned in order to trap the plug (4), which is split (4a) so as to be trapped elastically in the recess.

The pad is manufactured in any appropriate material that makes a flexible and elastic component, in particular such as the synthetic rubbers, polyurethanes, polyamides, the composite plastic/elastomer materials, or polyvinyl chloride.

The recess (5) is open at the side (5a) facing the tongue, so as to accommodate the part (2a) of the tongue which precedes the plug (4).

The underface (lc) of the pad is rendered adhesive so that it adheres to the skin.

The adhesive can be double-sided adhesive strip for example.

FIGS. 4 to 7 relate to implementation variants which are characterised in particular by the fact that:
the ribs (1a) in relief on the base (lb) of the pad form between them (FIGS. 4 to 7) at least one slot (7) and several channels, and lateral side-walls (11) are provided between the exits from these channels or slots onto the periphery of the pad, so as to retain the catheter when wound around the pad, on at least part of the perimeter of the pad.
the slot (7) and the channels (8-11) are positioned in two perpendicular directions;
the retaining side-walls (11) are raised (FIG. 5);
the retaining side-walls (11) are sloped upwards (FIG. 6) so as to retain the wound-on catheter;
the extremities (12) of the ribs (1a) which form the slot and the channels (7-11) project to the outside of the pad so as to retain beneath them the wound-on catheter on the periphery of the pad. Different options are thus offered to the user for the direction of emergence of the catheter at the exit from the pad (FIG. 7).
in the case of FIG. 7A, the catheter which exits from the hole (6) passes via a first channel (10) perpendicular to the slot (7), passes along the pad in one direction, until it reaches a second channel (9) located as an extension to the first channel (8), returns to the top of the hole, is turned through a right angle and emerges via the slot (7);
in the variant of FIG. 7B, the catheter passes along the pad to reach the channel (9) and pass via the channel (10) to cover the original branch already in place in this channel;
in the variant of FIG. 7C, the catheter, which has come out of the hole (6), has passed into the channel (10) and passes along the pad in the other direction, reaches the channel (8) aligned with the insertion slot (7), returns to the top of the hole and emerges via the insertion slot (7);
and in the variant of FIG. 7D, the catheter, instead of emerging via the insertion slot (7), is turned through a right angle and emerges via the second channel (9) which is perpendicular to the insertion slot.

In all of these cases, retention is finally performed by the plug (4), which is folded onto the catheter.

The invention is not limited to these implementation examples.

1. A device to secure onto the skin a catheter exiting from a cutaneous emergence site, which includes a flexible pad whose underside is rendered adhesive, and which presents a slot starting from the edge of the pad and ending at a hole which traverses the pad from side to side, this slot being sufficiently wide so that the pad can be slid laterally via the slot along the catheter exiting from the site until said hole can be positioned over the site, wherein said hole is located at the bottom of a recess that is present on the top surface of the pad and in that the pad has a pliable lateral tongue which includes a plug designed to be trapped elastically in said recess, on top of the catheter, when the tongue is bent onto the pad.

2. A device according to claim 1 in which said recess is circular and is open laterally on the side facing the tongue, so as to accommodate a part of the tongue which immediately precedes the said plug when the tongue is folded.

3. A device according to claim 1 in which said plug projects above the recess when it is trapped in the latter.

4. A device according to claim 1, in which said plug is split so as to be elastically deformable, and so that it will be trapped in the said recess.

5. A device according to claim 1, which includes a channel formed on the top of the pad in order to accommodate the catheter, by elastic trapping, at an exit from the hole.

6. A device according to claim 5, wherein said channel is on a downward slope as far as the edge of the pad in order to conduct the catheter until it makes contact with the skin.

7. A device according to claim 5 in which the channel extends in a straight line from the edge of the recess as far as the edge of the pad.

8. A device according to claim 5 in which the channel is located as an extension to said slot.

9. A device according to claim 5 in which the channel extends perpendicularly to said slot.

10. A device according to claim 1 in which said recess is formed in a thickened area of the pad.

11. A device according to claim 1 in which the pad includes other channels connecting the to the periphery of the pad.

12. A device according to claim 11 in which the slot and the channels are positioned in two perpendicular directions.

13. A device according to claim 5, wherein said pad has a bottom face comprising projecting ribs between which there are formed the slot and the channel or channels.

14. A device according to claim 13, wherein said ribs have extremities which extend beyond the bottom face of the pad and under which a catheter which is wound around the pad can pass.

15. A device according to claim 14 in which, between the exits from the slot and from the channels the base face of the pad has rims that project from the base face in order to retain the catheter laterally onto the periphery of the pad.

16. A device according to claim 15 in which the retaining rims comprise walls which are sloped upwards.

17. A device according to claim 1, in which said recess, said tongue and its plug are created by moulding with said pad in a synthetic resin.

18. A device according to claim 17 in which the pad is moulded with a resin from the group that includes the synthetic rubbers, polyurethanes, polyamides, and composite plastic/elastomer materials.
